(19) United States
(12) Patent Application Publication
(10) Pub. No.: US 2009/0169591 A1
(43) Pub. Date: Jul. 2, 2009

(54) MEDICAL ARTICLES FOR THE TREATMENT OF TUMORS

(75) Inventors: Goldi Kaul, Nyack, NY (US); John E. O'Gara, Ashland, MA (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/340,131

(22) Filed: Dec. 19, 2008

Related U.S. Application Data
(60) Provisional application No. 61/009,378, filed on Dec. 28, 2007.

(51) Int. Cl. A61K 9/00; A61K 47/32; A61K 38/16; A61K 31/4164; A61K 31/122; A61K 31/13; A61K 31/02; A61P 35/00

(52) U.S. Cl. 424/422; 514/772.4; 514/12; 514/398; 514/690; 514/644; 514/759; 514/6

(57) ABSTRACT

In accordance with one aspect of the invention, implantable and insertable medical articles are provided which are useful for the local treatment of tumors. These medical articles comprise one or more active agents that influence the local tumor environment in vivo, for example, decreasing the level of nutrients in the local tumor environment, inhibiting the utilization of nutrients in the local tumor environment, decreasing the amount of molecular oxygen and/or reactive oxygen species in the local tumor environment, or increasing the amount of molecular oxygen and/or reactive oxygen species in the local tumor environment. Other aspects of the invention pertain to methods of treatment that employ such medical articles. Still other aspects of the invention pertain to methods of making such medical articles.

### STATEMENT OF RELATED APPLICATIONS

This application claims the benefit of U.S. Provisional Patent Application Ser. No. 61/009,378, filed Dec. 28, 2007, entitled “Medical Articles For The Treatment of Tumors”, which is incorporated by reference herein in its entirety.

### FIELD OF THE INVENTION

The invention relates to medical articles, including methods for their manufacture and methods for their use in treating tumors.

### BACKGROUND OF THE INVENTION

Various medical articles are known which can be implanted or inserted into the vasculature for the treatment of various diseases and conditions, including the treatment of tumors. For example, the technique of embolization involves the therapeutic introduction of embolic agents, for example, embolic particles, into the circulation to occlude blood vessels, for instance, to cut off blood flow to a structure or organ. Permanent or temporary occlusion of blood vessels is desirable for managing various diseases, disorders and conditions, including the treatment of tumors.

In a typical embolization procedure, local anesthesia is first given over a common artery. The artery is then percutaneously punctured and a catheter is inserted and fluoroscopically guided into the area of interest. An angiogram may then be performed by injecting contrast agent through the catheter. An embolic agent is then deposited through the catheter. The embolic agent is chosen, for example, based on the size of the vessel to be occluded, the desired duration of occlusion, and/or the type of abnormality to be treated, among others factors. A follow-up angiogram is usually performed to determine the specificity and completeness of the arterial occlusion.

Various microspheres are currently employed to embolize blood vessels. These microspheres are usually introduced to the location of the intended embolization through microcatheters. Current commercially available embolic microspheres are commonly composed of biostable polymers. Materials used commercially for this purpose include polyvinyl alcohol (PVA), acetalized PVA (e.g., Contour® embolic agent, Boston Scientific, Natick, Mass., USA) and crosslinked acrylic hydrogels (e.g., Embospheres®, Biosphere Medical, Rockland, Mass., USA). Similar devices have been used in chemoembolization to increase the residence time of a therapeutic agent after delivery. In one specific instance, a therapeutic agent (doxorubicin) has been directly added to hydrogel microspheres (prepared from N-acrylamidoacetaldehyde derivatized polyvinyl alcohol copolymerized with 2-acrylamido-2-methylpropane sulfonate) such that the therapeutic agent can be released locally after delivery (e.g., DC Bead™ drug delivery chemoembolization system, Biocompatibles International plc, Farnham, Surrey, UK). Other examples of commercially available microspheres include glass microspheres with entrapped radioisotopes (e.g., ⁹⁰Y), in particular, TheraSpheres™, MDS Nordion, Ottowa, Canada and polymer microspheres that contain monomers that are capable of chelating radioisotopes (e.g., ⁹⁰Y), in particular, SIR-Spheres®, SIRTex Medical, New South Wales, Australia.

### SUMMARY OF THE INVENTION

In accordance with one aspect of the invention, implantable and insertable medical articles are provided which are useful for the local treatment of tumors. These medical articles comprise one or more active agents that influence the surrounding environment in vivo, for example, decreasing the level of nutrients in the local tumor environment, inhibiting the utilization of nutrients in the local tumor environment, decreasing the amount of molecular oxygen and/or reactive oxygen species in the local tumor environment, or increasing the amount of molecular oxygen and/or reactive oxygen species in the local tumor environment.

Other aspects of the invention pertain to methods of treatment that employ such medical articles.

Still other aspects of the invention pertain to methods of making such medical articles.

These and various additional aspects, embodiments and advantages of the present invention will become immediately apparent to those of ordinary skill in the art upon review of the Detailed Description and any claims to follow.

### DETAILED DESCRIPTION

As noted above, in one aspect of the invention, implantable and insertable medical articles are provided which are useful for the treatment of tumors. These medical articles comprise one or more active agents that influence the surrounding environment in vivo, for example, decreasing the level of nutrients in the local tumor environment, inhibiting the utilization of nutrients in the local tumor environment, decreasing the amount of molecular oxygen and/or reactive oxygen species in the local tumor environment, or increasing the amount of molecular oxygen and/or reactive oxygen species in the local tumor environment.

Examples of medical articles which may be employed for the treatment of tumors include medical articles for implantation or insertion into the vasculature, including stents, catheters, vascular grafts, embolic implants including embolic particles such as embolic microspheres, embolic materials that are injected as a fluid and solidify in the vasculature (e.g., glues and compositions that form gels in vivo), as well as coils, fibered coils, and occlusion balloons, among others.

Medical articles in accordance with the invention may be used to treat a variety of subjects, including vertebrate subjects, particularly humans and various warm-blooded animals, including pets and livestock.

Medical articles in accordance with the invention may be formed from a variety of materials, including polymeric materials, metallic materials, ceramic materials and hybrid articles (e.g., polymer coated metallic or ceramic articles, etc.), among others. Medical articles in accordance with the invention may be at least partially biostable. Medical articles in accordance with the invention may be at least partially bioresorbable. Medical articles in accordance with the invention may be at least partially porous. Medical articles in accordance with the invention may be at least partially nonporous.

Depending on the embodiment, the active agents in the medical articles of the invention may be releasable or non-releasable. Release may occur spontaneously in vivo or after in vivo activation, for example, activation based on the application of heat, light or other energy or activation using a chemical agent that causes release.

Among other characteristics, the active agents may be, for example, hydrophobic, hydrophilic or amphiphilic, and they may be charged or uncharged.

The active agent may be disposed at the surface of the medical article (e.g., covalently or non-covalently bound to the surface of the article, which surface may be porous or nonporous, with porous articles exhibiting enhanced surface area). The active agent may be disposed within the medical article (e.g., covalently or non-covalently bound within a polymeric, ceramic or metallic matrix, physically entrapped or encapsulated within the article via a polymeric, ceramic or metallic barrier layer, etc.). The active agent may be both disposed at the surface and within the medical article.

Non-covalent interactions that may be employed to temporarily or permanently bind an active agent to a medical article include specific and non-specific non-covalent interactions such as those based on van der Waals forces, hydrophobic interactions and/or electrostatic interactions (e.g., charge-charge interactions, charge-dipole interactions, and dipole-dipole interactions, including hydrogen bonding). Some examples of specific non-covalent interactions include π-π stacking, binding based on the formation of multiple hydrogen bonds, binding based on the formation of complexes and/or coordinative bonds (e.g., metal ion chelation, etc.), binding based on antibody-antigen interactions, also sometimes referred to as antibody-hapten interactions, protein-small molecule interactions (e.g., avidin/streptavidin-biotin binding), and protein-protein interactions, among others. Specific chemical entities (e.g., binding ligands) may be covalently attached to the medical articles in order to create specific noncovalent interactions between the medical article and the active agent.

Embodiments of the invention pertaining to embolic implants, specifically, embolic particles, will generally be discussed herein for ease of illustration, but it will be understood that the embodiments described in conjunction with embolic particles are clearly applicable to other medical articles including those described above.

With respect to embolic particles in accordance with the invention, such particles may vary widely in shape. In certain embodiments, they are substantially spherical, for example, having the form of a perfect (to the eye) sphere or the form of a near-perfect sphere such as a prolate spheroid (a slightly elongated sphere) or an oblate spheroid (a slightly flattened sphere), among other possibilities. In other embodiments they may be in the form of another regular geometry (e.g., cylindrical, etc.) or an irregular geometry. In embodiments where the particles are substantially spherical, at least half of the particles (50% or more, for example, from 50% to 75% to 90% to 95% or more of a particle sample) may have a sphericity of 0.8 or more (e.g., from 0.80 to 0.85 to 0.9 to 0.95 to 0.97 or more). The sphericity of a collection of particles can be determined, for example, using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures the particles in an image. The sphericity of a particle, which is computed as Da/Dp (where Da=√(4A/π); Dp=P/π; A=pixel area; P=pixel perimeter), is a value from zero to one, with one representing a perfect circle.

The embolic particles of the invention can vary in size, with typical longest linear cross-sectional dimensions (e.g., for a sphere, the diameter) ranging, for example, from 40 to 100 to 150 to 250 to 500 to 750 to 1000 to 1500 to 2000 to 2500 to 5000 microns (μm).

For a collection of particles, the arithmetic mean maximum for the group typically ranges, for example, from 40 to 100 to 150 to 250 to 500 to 750 to 1000 to 1500 to 2000 to 2500 to 5000 microns (μm). The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the maximum dimensions (e.g., the diameter, for a sphere) of all of the particles in the group by the number of particles in the group.

In some embodiments, at least 95 vol % of the particles within a group have longest linear cross-sectional dimensions between 40 μm and 5000 μm. For example, where the particles are spherical at least 95 vol % of the particles may have diameters between 40 μm and 5000 μm. More particularly, depending on the embodiment, at least 95 vol % of the particles within a group may have longest linear cross-sectional dimensions between any two of the following dimensions: 40, 100, 150, 250, 500, 750, 1000, 1500, 2000, 2500 and 5000 microns.

In some embodiments, the particles are porous particles. As used herein a “porous particle” is a particle that contains pores, which may be observed, for example, by viewing the pores using a suitable microscopy technique such as scanning electron microscopy. Pore size may vary widely, ranging from 1 micron or less to 2 microns to 5 microns to 10 microns to 25 microns to 50 microns to 100 microns or more. Pores can come in a wide range of shapes. In some embodiments, the particles comprise a porous surface layer disposed over a non-porous core. In other embodiments, pores are present throughout the interior of the particles.

As indicated above, medical articles in accordance with the invention (including embolic particles, among others) comprise one or more active agents that influence the surrounding environment in vivo, for example, decreasing the level of nutrients in the surrounding environment, inhibiting the utilization of nutrients in the surrounding environment, decreasing the amount of oxygen and/or reactive oxygen species in the surrounding environment, or increasing the amount of oxygen and/or reactive oxygen species in the surrounding environment. Examples of reactive oxygen species include superoxide (O₂⁻), hydrogen peroxide (H₂O₂), hydroxyl radical, and peroxynitrite, among others.

For example, various aspects of the invention employ novel embolic particles for treating tumors, including hypervascularized tumors, wherein the intra-arterial administration of embolic particles not only causes a physical blockade in the vasculature to cause emboli, but also affects the level of nutrients, molecular oxygen and/or reactive oxygen species in the local environment of the tumors.

In some aspects of the invention, embolic particles are provided with one or more active agents which can deplete the local in vivo environment (e.g., the vasculature associated with a tumor) of nutrients. The embolic particle is administered such that it not only blocks the vasculature in the form of an emboli (thereby depleting the tumor's blood supply), but also such that it promotes tumor kill by depleting the local environment of nutrients. Examples of the nutrients required for cell growth include (a) amino acid containing species such as peptides and proteins (including glycoproteins, metalloproteins, lipoproteins), (b) carbohydrate containing species such as monosaccharides, oligosaccharides containing from 2 to 30 monosaccharide units and higher polysaccharides containing more than 30 monosaccharide units, including complex carbohydrates (e.g., glycoproteins), and (c) lipid containing species including triglycerides and other fatty-acid-based molecules. The depletion of nutrients assists in starving the tumor.

Nutrient depletion may be accomplished in several ways. In some embodiments, the embolic particles scavenge the nutrient from the local environment, sequestering the nutrient within the embolic particles. In some embodiments, the embolic particles release an active agent that converts nutrients in the local environment into a form which cannot be used by the tumor cells.

In this regard, the successful use of an enzyme, asparaginase, to selectively deplete serum levels of its substrate, asparagine, and thus to produce antitumor effects in acute lymphocytic leukemia (J. R. Beitino et al., “Nutritional Factors in the Design of More Selective Antitumor Agents,” Cancer Research 29, Dec. 1969, 2417-2421) indicates that various selective antitumor responses may be obtained by depletion of normal nutrients. The rationale for such an approach lies in the fact that certain tumors appear to have a greater requirement for preformed substances (some of which may not be considered essential) than do normal host tissues.

In some embodiments, the embolic particles act to scavenge amino acid containing species such as peptides and proteins or act to release an active agent which converts the nutrient into a form which cannot be used by the tumor cells. For example, the embolic particles may be provided with affinity ligands which can bind to proteins to remove them from local environment. Examples of affinity ligands include immobilized Cibacron Blue or related chlorotriazine dye, bacterial protein A or G, single-chain antibody fragments, ionic ligands such as heparin and boronic acid, as well as immobilized glutathione. Protein ligands can attach themselves to protein receptors such as antibodies, enzymes, hormone receptors, integral membrane proteins, and other proteins. Affinity ligands include antigens, enzymatic inhibitors, hormone agonists, antibodies, prodrugs, small binding peptides and various small molecule drugs, among others. Such ligands may be incorporated into embolic particles using a number of synthetic and/or coupling methods well known to those skilled in the art.

As another example, the embolic particles can be provided with an active agent that is released into the local environment to covert amino acid containing species into a form which cannot be used by the tumor cells. Examples of such active agents include agents that chemically induce the coagulation of the proteins. Specific examples of protein coagulants include sodium polyphosphate, ferric chloride, lignin, and sodium lignosulfonate. See V. Vandergrift and A. L. Ratermann, J. Agric. Food Chem. 1979, 27(6) 1252-1256.

In some embodiments, the embolic particles scavenge carbohydrate containing species such as glucose or other sugar-based molecules or release an active agent which converts the nutrient into a form which cannot be used by the tumor cells. For example, the embolic particles may be provided with phenylboronic acid (PBA) ligands which can bind to the cis-diol groups of various carbohydrate containing species, including sugars such as glucose and other more complex carbohydrates, thereby removing these species from the local environment. PBA groups may be incorporated into embolic particles using a number of synthetic and/or coupling methods well known to those skilled in the art. See, e.g., T. Hoare and R. Pelton, Macromolecules 2007, 40(3), 670-678. As another example, the embolic particles may be provided with an active agent that is released into the local environment to convert carbohydrate containing species into a form which cannot be used by the tumor cells.

In this regard, adequate carbohydrates and amino acids given simultaneously are found to enhance both host maintenance and tumor growth. However, an isocaloric, isonitrogenous, intravenous diet providing non-nitrogenous calories as fat are found to promote host maintenance equivalent to carbohydrate-based TPN (total parenteral nutrition), but without tumor stimulation. G. P. Buzby et al., “Host-tumor interaction and nutrient supply,” Cancer, Volume 45, Issue 12, Pages 2940-2948. Thus, in some embodiments of the invention, the effects of embolic particles that reduce the availability carbohydrate containing species (e.g., by scavenging, conversion, inhibiting utilization, etc.) may be enhanced through the use of a suitable parenteral nutrition regimen. Conversely, parenteral nutrition may be used to provide tumor stimulation at appropriate times to increase sensitivity to phase-specific antineoplastic therapy.

In some embodiments, the embolic particles scavenge lipid containing species such as triglycerides or other fatty acid-based molecules or release an active agent which converts these nutrients into a form which cannot be used by the tumor cells. For example, embolic materials may be provided which possess hydrophobic groups (or distinct hydrophobic regions) which sequester the triglycerides or other fatty acid-based molecules through a hydrophobic interaction. As another example, the embolic materials may be provided with positively charged ion exchange groups which bind with the negatively charged carboxylate group of the fatty acids. Hydrophobic or ion exchange groups may be incorporated into embolic particles using a number of synthetic and/or coupling methods well known those skilled in the art. In one specific example, polymers comprising hydrophobic and/or ion exchange groups are grafted onto the embolic particles and/or provided within the embolic particles. As another example, the embolic particles can be provided with an active agent that is released into the local environment to covert lipid-containing-species into a form which cannot be used by the tumor cells.

In other embodiments, the embolic particles scavenge essential minerals and metals or release an active agent which converts these nutrients into a form which cannot be used by the tumor cells. For example, embolic materials may be provided which possess affinity ligands (e.g., chelating groups) which sequester the essential minerals and metals (e.g., through a chelation interaction). Examples of such affinity ligands include immobilized iminodiacetic acid. In this regard, depletion of either zinc or magnesium has been found to significantly inhibit tumor growth in rats. However, the magnesium depletion resulted in a greater degree of inhibition with less loss of carcass weight. In both studies, depleted tumor-bearing rats maintained weights of vital organs such as liver, kidney and heart. Moreover, there were significant decreases in tumor organ ratios in deficient groups. B. J. Mills et al., “Inhibition of Tumor Growth by Magnesium Depletion of Rats,” Journal of Nutrition, Vol. 114, No. 4, April 1984, pp. 739-745 and B. J. Mills et al., “Inhibition of Tumor Growth by Zinc Depletion of Rats,” Journal of nutrition, Vol. 114, No. 4, April 1984, pp. 746-752.

In other embodiments, the embolic particles of the invention release active agents that inhibit the utilization of nutrients by the tumor cells. In accordance with certain of these embodiments, embolic particles are provided which release antimetabolites that can act to provide dietary deprivation of the tumor. For example, L-glutamine is a substrate taken up by various tumors. In certain embodiments, the embolic particles of the invention may be provided, which release L-glutamine antimetabolites such as azaserine or diazooxonorleucine, among other possibilities, thereby interfering with glutamine metabolism within the tumor.

In some embodiments, an embolic particle (or a combination of two or more particles, each containing a different active agent) is employed which can act to deplete and/or inhibit utilization of a combination of two or more nutrients (e.g., amino acid containing species, carbohydrate containing species, lipid-containing species, metals, etc.).

In some embodiments, an embolic particle (or a combination of two or more embolic particles, each containing a different active agent) is employed, which can act to deplete and/or inhibit utilization of one or more nutrients and which contains one or more anti-tumor agents (in releasable or non-releasable form), such as one or more chemotherapy agents.

Specific examples of chemotherapy agents for use in the particles of the invention may be selected from suitable members of the following: radioisotopes (e.g., ⁹⁰Y, ³²P, ¹⁸F, ¹⁴⁰La, ¹⁵³Sm, ¹⁶⁵Dy, ¹⁶⁶Ho, ¹⁶⁹Er, ¹⁶⁹Yb, ¹⁷⁷Lu, ¹⁸⁶Re, ¹⁸⁸Re, ¹⁰³Pd, ¹⁹⁸Au, ¹⁹²Ir, ⁹⁰Sr, ¹¹¹In or ⁶⁷Ga), which may be covalently bound or non-covalently bound to another species, antineoplastic/antiproliferative/ anti-mitotic agents including antimetabolites such as folic acid analogs/antagonists (e.g., methotrexate, etc.), purine analogs (e.g., 6-mercaptopurine, thioguanine, cladribine, which is a chlorinated purine nucleoside analog, etc.) and pyrimidine analogs (e.g., cytarabine, fluorouracil, etc.), alkaloids including taxanes (e.g., paclitaxel, docetaxel, etc.), alkylating agents such as alkyl sulfonates, nitrogen mustards (e.g., cyclophosphamide, ifosfamide, etc.), nitrosoureas, ethylenimines and methylmelamines, other aklyating agents (e.g., dacarbazine, etc.), antibiotics and analogs (e.g., daunorubicin, doxorubicin, idarubicin, mitomycin, bleomycins, plicamycin, etc.), antiestrogens (e.g., tamoxifen), antiandrogens (e.g., flutamide), platinum complexes (e.g., cisplatin, carboplatin, etc.), antineoplastic enzymes (e.g., asparaginase, etc.), agents affecting microtubule dynamics (e.g., vinblastine, vincristine, colchicine, Epo D, epothilone), caspase activators, proteasome inhibitors, angiogenesis inhibitors (e.g., statins such as endostatin, cerivastatin and angiostatin, squalamine, etc.), olimus family agents including sirolimus, everolimus, tacrolimus and zotarolimus, etoposides, as well as many others (e.g., hydroxyurea, flavopiridol, procarbizine, mitoxantrone, campothecin, etc.), various pharmaceutically acceptable salts and derivatives (e.g., esters, etc.) of the foregoing, and combinations of the foregoing, among other agents.

In some aspects of the invention, embolic particles are provided which contain one or more active agents which can affect the amount of molecular oxygen and/or reactive oxygen species (e.g., superoxide, hydrogen peroxide, hydroxyl radical, peroxynitrite, etc.) in the surrounding environment in vivo.

For instance, in some embodiments, embolic particles are provided with one or more active agents that can reduce the amount of molecular oxygen in the surrounding environment in vivo. By depleting the resident tumor of its oxygen supply (and causing local hypoxic conditions), tumor kill may be achieved.

Hypoxic conditions may be achieved, for example, by providing the embolic particles with active agents that consume oxygen in the local environment, thus causing local hypoxic conditions in the vicinity of the tumor. Such agents may be disposed on and/or within the particles in a releasable or non-releasable fashion.

For example, in some embodiments, the embolic particles are provided with oxygen scavenging agents such as a dithionite, bisulfite, metabisulfite or sulfite of an alkali metal or of ammonium, for example, sodium dithionite, or other oxygen scavenging agents such as L-cysteine, N-acetyl-L-cysteine, Na₂S, FeS, sodium thioglycolate, sodium pyruvate, or Oxyrase®. In some embodiments, the embolic particles are provided with species that can be converted in vivo (e.g., by application of light, heat or another form of energy) into an oxygen scavenging agent. In certain of these embodiments, the applied energy not only results in oxygen scavenging, but also results in the formation of a new, more-toxic species. For example, embolic particles in accordance with the invention may be provided with photosensitizing agents that, upon exposure to light of a specific wavelength, scavenge oxygen and produce toxic species such as singlet oxygen, which are able to kill tumor cells. Examples of such agents include porfimer sodium (Photofrin®), aminolevulinic acid (ALA), 2-[1-hexyloethyl]-2-devinyl-pyropheophorbidea (HPPH or photochlor), benzoporphyrin derivative monoacid ring A (BPD-MA or verteporfin), meta-tetra hydroxyphenyl chlorine (temoporfin), and motexafin lutetium. Such agents may be disposed on and/or within the particles in a releasable or non-releasable fashion.

For instance, embolic particles containing Photofrin® may be administered to a subject using a suitable embolic guideline protocol and the area of the embolism that is produced may be subjected to photodynamic therapy (PDT) using light at wavelength of 630 nm. This light causes the activation of Photofrin®, which in the presence molecular oxygen in the area of the tumor results in the formation of singlet oxygen, a highly reactive state of oxygen that damages lipids, nucleic acids and other cellular components, leading to cell death. These embolic particles may thus provide a three-pronged therapy wherein the tumor kill is achieved not only by an embolic effect, but also by generation of reactive oxygen species and by hypoxia, as the PDT induces hypoxia due to oxygen depletion during the photochemical reaction wherein the reactive oxygen species is produced.

In addition to active agents that consume molecular oxygen in the local environment, thereby promoting local hypoxic conditions in the vicinity of the tumor, embolic particles in accordance with the invention may also contain one or more active agents that undergo bioreductive activation and become more toxic under hypoxic conditions. Examples of such agents include (a) nitroimidazoles, such as misonidazole, metronidazole, benznidazole, desmethylmisonidazole, etanidazole, pimonidazole, nimorazole, omidazole, and RSU 1069, (b) quinones (including quinones based on the indolequinone nucleus) such as mitomycin C, porfiromycin (N-methylmitomycin C), E09 (indoloquinone), diaziquone, triaziquone, and carbazilquinone, and (c) aromatic and aliphatic N-oxides (including benzotriazine di-N-oxides) such as tirapazamine and AQ4N [1,4-bis[2-(dimethylamino-N-oxide)ethyl]amino 5,8-di-hydroxyanthracene-9,10-dione]. Such agents may be disposed on and/or within the particles in a releasable or non-releasable fashion.

For instance, N-oxides such as tirapazamine represent a new class of hypoxia-selective cytotoxins that under hypoxic conditions undergo enzymatic reduction and form highly reactive radicals, which can kill cells by causing DNA damage that leads to chromosomal aberrations. This hypoxia-induced ability to target cancer cells is independent of the P53 status of the cell. Thus, in certain embodiments, such N-oxides may be disposed on and/or within the particles in a releasable fashion.

Reactive oxygen species have been reported to be increased in malignant cells in part as a result of oncogene signaling via the NADPH oxidase complex and by hypoxia-related mitochondrial ROS. See, e.g., J. P. Fruehauf and F. L. Meyskans Jr., Clin. Cancer Res. 13 (2007) 789-794 and the references cited therein. Increased levels of reactive oxygen species in turn contribute to enhanced cell proliferation and apoptosis suppression. Id.

Thus, in some embodiments of the invention, embolic particles are provided with active agents that are capable of reducing the amount of reactive oxygen species in the surrounding environment. Examples of such active agents include, for example, superoxide dismutases (SOD), SOD mimetics, glutathione peroxidase, and catalase, among others. Such agents may be disposed on and/or within the particles in a releasable or non-releasable fashion.

In other embodiments of the invention, embolic particles are provided which contain one or more active agents that can increase the amount of molecular oxygen in the surrounding environment in vivo. For example, embolic particles may the provided that function as molecular oxygen carriers. Examples of such particles include those that have oxygen carrying capacity, for example, by physical encapsulation or by covalent or non-covalent binding of molecular oxygen carriers such as perfluorocarbons or any of a variety of hemoglobins such as purified hemoglobin, recombinant hemoglobin, polymerized hemoglobin, cross-linked hemoglobin, or other forms of hemoglobin. Such agents may be disposed on and/or within the particles in a releasable or non-releasable fashion. These embolic particles may be used, for example, in conjunction with radiation therapy and other current therapies that rely on the formation of harmful free radical species to damage cells. Increasing the oxygen levels in the vicinity of the cancer cells renders the cells more vulnerable to such therapies.

In still other embodiments of the invention, embolic particles are provided which contain one or more active agents that are capable of increasing the amount of reactive oxygen species in the surrounding environment. Elevated levels of reactive oxygen species have been reported to cause apoptosis by triggering mitochondrial permeability transition pore opening and release of proapoptotic factors. See, e.g., J. P. Fruehauf and F. L. Meyskans Jr., supra, and the references cited therein.

For example, embolic particles may be provided which contain one or more active agents that produce reactive oxygen species (e.g., superoxide, hydrogen peroxide, hydroxyl radical, peroxynitrite, etc.) directly in vivo. Such agents may be disposed on and/or within the particles in a releasable or non-releasable fashion. For instance, sodium percarbonate and sodium perborate degrade in vivo to produce hydrogen peroxide.

Embolic particles may also be provided which contain one or more active agents that interfere with reactive oxygen species scavenging in order to elevate local levels of reactive oxygen species. Such agents may be disposed on and/or within the particles in a releasable or non-releasable fashion, depending on the embodiment. Examples of active agents that promote accumulation of reactive oxygen species in this manner include agents that deplete glutathione, such as buthionine sulfoximine and arsenic trioxide, agents that chelate copper (and thus inhibit superoxide dismutases including Cu,Zn—SOD), such as disulfiram and ATN224, and agents that inhibit thioredoxin, including flavanols, such as quercetin, motexafin gadolinium and chemotherapy agents that can inhibit thioredoxin including melphalan, carmustine, cisplatin, and oxaliplatin.

In some embodiments, an embolic particle (or a combination of two or more particles, each having a different active agent) is employed which contain (a) one or more agents that can affect the amount of oxygen and/or reactive oxygen species in the surrounding environment (see above) and (b) one or more additional agents, for example anti-tumor agents such as chemotherapy agents (see above) or vasoactive agents (e.g., vasoconstrictors or vasodilators) such as epinephrine, isoproterenol or chlorpromazine, among others.

In accordance with one aspect of the present invention, a method is provided which comprises (a) determining the oxygen level within a given tumor (e.g. tumor hypoxia level) for example, using an electrochemical probe that produces an electrical current depending on the amount of oxygen present in the tissue, binding of [3H] misonidazole aided positron emission tomography, or by generating an Eppendorf pO₂ polarographic microelectrode histograph, which requires direct insertion of a needle through multiple tracts of a tumor to obtain multiple samplings of tumor oxygen tension and (b) depending on the tumor type and desired therapy regimen, administering either embolic particles that increase oxygen levels or embolic particles that decrease oxygen levels.

In accordance with another aspect of the present invention, a method is provided which comprises administering embolic particles that increase molecular oxygen levels, followed by administering embolic particles that cause hypoxia. In some embodiments, chemotherapy or radiation therapy is applied after administering the particles that increase local molecular oxygen levels. Such a method may be used, for example, to first elevate local oxygen levels (which may, for example, promote chemotherapeutic kill effect and/or radiation kill effect on the peripheral tumor cells that are the actively dividing and metastasizing), followed by the creation of tumor hypoxia (which may, for example, remove the highly dense necrotic and hypoxic tumor core).

In accordance with another aspect of the present invention, a method is provided which comprises administering embolic particles that cause hypoxia, followed by administering embolic particles that increase molecular oxygen levels. In some embodiments, chemotherapy or radiation therapy is applied after administering the particles that increase oxygen molecular oxygen levels.

In accordance with yet another aspect of the present invention, a method is provided which comprises concurrently administering embolic particles that promote tumor kill by photodynamic therapy (which depletes local oxygen) and embolic particles that increase molecular oxygen levels in vivo. In this way, oxygen carrying particles may function as reservoirs of molecular oxygen, which promotes tumor kill by providing the oxygen that is needed for photodynamic therapy (which oxygen is depleted during photodynamic therapy).

In certain embodiments, embolic particles in accordance with the present invention may include one or more radiopaque materials, materials that are visible under magnetic resonance imaging (MRI-visible materials), ferromagnetic materials, and/or ultrasound contrast agents. These materials can, for example, be physically encapsulated/entrapped within the particles or covalently to non-covalently associated with the particles. Various radiopaque materials, MRI-visible materials, ferromagnetic materials, and contrast agents are described, for example, in Pub. No. US 2004/0101564 A1 to Rioux et al.

In certain embodiments, the embolic particles of the present invention are polymeric particles. As used herein a “polymeric particle” is one that contains polymers, for example, from 50 wt % or less to 75 wt % to 90 wt % to 95 wt % to 97.5 wt % to 99 wt % or more polymers.

As used herein, “polymers” are molecules that contain multiple copies of one or more types of constitutional units, commonly referred to as monomers. The number of monomers/constitutional units within a given polymer may vary widely, ranging, for example, from 5 to 10 to 25 to 50 to 100 to 1000 to 10,000 or more constitutional units. As used herein, the term “monomers” may refer to the free monomers and those that are incorporated into polymers, with the distinction being clear from the context in which the term is used.

Polymers for use in the present invention can have a variety of architectures, including cyclic, linear and branched architectures. Branched architectures include star-shaped architectures (e.g., architectures in which three or more chains emanate from a single branch point), comb architectures (e.g., architectures having a main chain and a plurality of side chains, such as graft polymers), dendritic architectures (e.g., arborescent and hyperbranched polymers), and networked architectures (e.g., crosslinked polymers), among others.

Polymers containing a single type of monomer are called homopolymers, whereas polymers containing two or more types of monomers are referred to as copolymers. The two or more types of monomers within a given copolymer may be present in any of a variety of distributions including random, statistical, gradient and periodic (e.g., alternating) distributions, among others. One particular type of copolymer is a “block copolymer,” which is a copolymer that contains two or more polymer chains of different composition, which chains may be selected from homopolymer chains and copolymer chains (e.g., random, statistical, gradient or periodic copolymer chains).

Polymeric particles in accordance with the invention may be biostable or bioresorbable. As used herein, a polymeric particle is “bioresorbable” if it disintegrates in vivo due to one or more mechanisms such as dissolution, biodegradation, and so forth. On the other hand, a polymeric particle is “biostable” if it does not disintegrate in vivo.

As used herein, a polymer is “biodegradable” if it undergoes bond cleavage along the polymer backbone in vivo, regardless of the mechanism of bond cleavage (e.g., enzymatic breakdown, hydrolysis, oxidation, etc.).

In some embodiments of the invention, the polymeric particles are hydrogel particles. As used herein, a “hydrogel” is a crosslinked hydrophilic polymer (e.g., a polymer network) which swells when placed in water or biological fluids, but remains insoluble due to the presence of crosslinks, which may be, for example, physical, chemical, or both. For instance, a hydrogel particle in accordance with the invention may undergo swelling in water such that its longest linear cross-sectional dimension (e.g., for a sphere, the diameter) increases by 5% or less to 10% to 15% to 20% to 25% or more. In some instances, the insolubility of the hydrogel is not permanent, and the particles biodisintegrate in vivo.

Specific polymers for as use in accordance with the invention may be selected, for example, from suitable members of the following, among others: polycarboxylic acid homopolymers and copolymers including polyacrylic acid, polymethacrylic acid, ethylene-methacrylic acid copolymers and ethylene-acrylic acid copolymers, where some of the acid groups can be neutralized with either zinc or sodium ions (commonly known as ionomers); acetal homopolymers and copolymers; acrylate and methacrylate homopolymers and copolymers (e.g., n-butyl methacrylate); cellulosic homopolymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene homopolymers and copolymers; polyimide homopolymers and copolymers such as polyether block imides, polyamidimides, polyesterimides, and polyetherimides; polysulfone homopolymers and copolymers including polyarylsulfones and polyethersulfones; polyamide homopolymers and copolymers including nylon 6,6, nylon 12, polycaprolactams, polyacrylamides and polyether block amides; resins including alkyd resins, phenolic resins, urea resins, melamine resins, epoxy resins, allyl resins and epoxide resins; polycarbonate homopolymers and copolymers; polyacrylonitrile homopolymers and copolymers; polyvinylpyrrolidone homopolymers and copolymers (cross-linked and otherwise); homopolymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene (SIBS) block copolymers such as those disclosed in U.S. Pat. No. 6,545,097 to Pinchuk), poly[(styrene-co-p-methylstyrene)-b-isobutylene-b-(styrene-co-p-methylstyrene)] (SMIMS) triblock copolymers described in S. J. Taylor et al., Polymer 45 (2004) 4719-4730; polyphosphonate homopolymers and copolymers; polysulfonate homopolymers and copolymers, for example, sulfonated vinyl aromatic polymers and copolymers, including block copolymers having one or more sulfonated poly(vinyl aromatic) blocks and one or more polyalkene blocks, for example, sulfonated polystyrene-polyolefin-polystyrene triblock copolymers such as the sulfonated SEBS copolymers described in U.S. Pat. No. 5,840,387, and sulfonated versions of SIBS and SMIMS, which polymers may be sulfonated, for example, using the processes described in U.S. Pat. No. 5,840,387 and U.S. Pat. No. 5,468,574, among other sulfonated block copolymers; polyvinyl ketones, polyvinylcarbazoles, and polyvinyl esters such as polyvinyl acetates; polybenzimidazoles; polyalkyl oxide homopolymers and copolymers including polyethylene oxides (PEO); polyesters including polyethylene terephthalates and aliphatic polyesters such as homopolymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether homopolymers and copolymers including polyarylethers such as polyphenylene ethers, polyether ketones, polyether ether ketones; polyphenylene sulfides; polyisocyanates; polyolefin homopolymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated homopolymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone homopolymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof, examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; polyamine and polyimine homopolymers and copolymers; biopolymers, for example, polypeptides including anionic polypeptides such as polyglutamate and cationic polypeptides such as polylysine, proteins, polysaccharides, and fatty acids (and esters thereof), including fibrin, fibrinogen, collagen, elastin, chitosan, gelatin, starch, glycosaminoglycans such as hyaluronic acid; as well as further copolymers, derivatives (e.g., esters, etc.) and mixtures of the foregoing.

Examples of hydrophilic polymers for as use in the invention, not necessarily exclusive of those set forth above, may be selected from suitable members of the following, among many others: homopolymers and copolymers of acrylic acid, methacrylic acid, acrylamides including N-alkylacrylamides, alkylene oxides such as ethylene oxide and propylene oxide, vinyl alcohol, vinyl pyrrolidone, ethylene imine, ethylene amine, acrylonitrile and vinyl sulfonic acid, amino acids such as lysine and glutamic acid and maleic anhydride, hydrophilic polyurethanes, proteins, collagen, cellulosic polymers such as methyl cellulose and carboxymethyl cellulose, dextran, carboxymethyl dextran, modified dextran, alginic acid, pectinic acid, hyaluronic acid, chitin, pullulan, gelatin, gellan, xanthan, starch, carboxymethyl starch, chondroitin sulfate, guar, and further copolymers, derivatives and mixtures of the foregoing. Many of these polymers may be physically crosslinked, chemically crosslinked, or both to form hydrogels.

Examples of biodegradable polymers, not necessarily exclusive of those set forth above, may be selected from suitable members of the following, among many others: (a) polyester homopolymers and copolymers such as polyglycolide, poly-L-lactide, poly-D-lactide, poly-D,L-lactide, poly(beta-hydroxybutyrate), poly-D-gluconate, poly-L-gluconate, poly-D,L-gluconate, poly(epsilon-caprolactone), poly(delta-valerolactone), poly(p-dioxanone), poly(trimethylene carbonate), poly(lactide-co-glycolide) (PLGA), poly(lactide-co-delta-valerolactone), poly(lactide-co-epsilon-caprolactone), poly(lactide-co-beta-malic acid), poly(lactide-co-trimethylene carbonate), poly(glycolide-co-trimethylene carbonate), poly(beta-hydroxybutyrate-co-beta-hydroxyvalerate), poly[1,3-bis(p-carboxyphenoxy)propane-co-sebacic acid], and poly(sebacic acid-co-fumaric acid), among others, (b) poly(ortho esters) such as those synthesized by copolymerization of various diketene acetals and diols, among others, (c) polyanhydrides such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3-bis(p-carboxyphenoxy)propane anhydride] and poly[1,3-bis(p-carboxyphenoxy)hexane anhydride], among others; and (d) amino-acid-based polymers including tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and a diphenol selected, for instance, from ethyl, butyl, hexyl, octyl and bezyl esters of desaminotyrosyl-tyrosine), and tyrosine-, leucine- and lysine-based polyester-amides; specific examples of tyrosine-based polymers include includes polymers that are comprised of a combination of desaminotyrosyl tyrosine hexyl ester, desaminotyrosyl tyrosine, and various di-acids, for example, succinic acid and adipic acid, for example, tyrosine-derived ester-amides such as the TyRx 2,2 family of polymers, available from TyRx Pharma, Inc., Monmouth Junction, N.J., USA, among others, as well as further copolymers, derivatives and mixtures of the foregoing.

Polymeric particles for use in the invention may be formed by any suitable particle forming method, including emulsion/solvent evaporation methods, precipitation methods, and droplet solidification methods, among many others.

The following discussion pertains to the formation of polymeric particles from polyols such as polyvinyl alcohol (PVA) for purposes of further illustrating the invention, but the invention is clearly not so-limited.

The monomer of PVA (vinyl alcohol), does not exist in a stable free form, due to keto-enol rearrangement with its tautomer (acetaldehyde). Typically, PVA is produced by the polymerization of a vinyl ester, such as vinyl acetate, to form a polyvinyl ester such as polyvinyl acetate (PVAc). Then the polyvinyl ester is subjected to hydrolysis to convert the ester groups to hydroxyl groups. The hydrolysis reaction, however, does not typically go to completion, resulting in polymers with a certain degree of hydrolysis that depends on the extent of reaction. Thus, PVA is generally a copolymer of vinyl alcohol

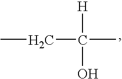

monomers and vinyl ester monomers, typically, vinyl acetate monomers

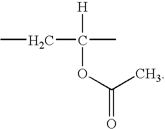

Commercial PVA grades are available with varying degrees of hydrolysis including grades with high degrees of hydrolysis (above 98.5%). The degree of hydrolysis (or, conversely, the ester group content) of the polymer has an effect on its chemical properties, crystallizability, and solubility, among other properties. For example, degrees of hydrolysis and polymerization are known to affect the solubility of PVA in water, with PVA grades having high degrees of hydrolysis being known to have reduced solubility in water relative to those having low degrees of hydrolysis. For further information on PVA (as well as PVA hydrogels), see, e.g., C. M. Hassan et al., “Structure and Applications of Poly(vinyl alcohol) Hydrogels Produced by Conventional Crosslinking or by Freezing/Thawing Methods,” Adv. Polym. Sci., 153, 37-65 (2000) and N. A. Peppas et al., “Hydrogels in Biology and Medicine: From Fundamentals to Bionanotechnology”, Adv. Mater., 18, 1345-1360 (2006).

As noted above, hydrogels are crosslinked hydrophilic polymers (e.g., polymer networks) which swell when placed in water or biological fluids, but remain insoluble due to the presence of crosslinks, which may be, for example, physical, chemical, or a combination of both.

Polyols such as PVA can be crosslinked, for example, through the use of chemical crosslinking agents. Some of the common chemical crosslinking agents that have been used for polyol hydrogel preparation include glutaraldehyde, acetaldehyde, formaldehyde, and other monoaldehydes. In the presence of an acid (e.g., sulfuric acid, acetic acid, etc.) these crosslinking agents form acetal bridges between the pendant hydroxyl groups found on the polyol chains. For example, acetal formation may link two alcohol moieties together according to the following scheme:

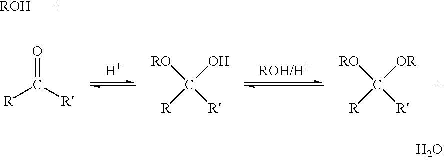

where R and R′ are organic groups. For species with multiple hydroxyl groups, including polyols such as PVA, two hydroxyl groups within the same molecule may react according to the following scheme:

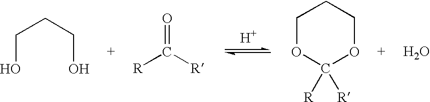

As noted in Pub. No. US 2003/0185895 to Lanphere et al., in certain instances, the reaction of PVA with an aldehyde (formaldehyde) in the presence of an acid is primarily a 1,3-acetalization:

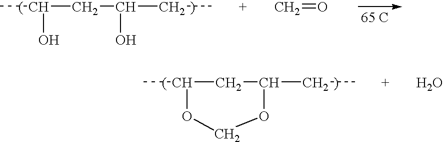

Such intra-chain acetalization reaction can be carried out with relatively low probability of inter-chain crosslinking. Since the reaction proceeds in a random fashion, there will be leftover —OH groups that do not react with adjacent groups. Moreover, the residual vinyl ester groups do not take part in the above reactions. Thus, PVA crosslinked in this fashion can be considered a copolymer of the following monomers: vinyl alcohol

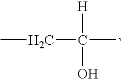

monomers, vinyl ester monomers, typically vinyl acetate

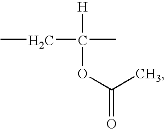

monomers and vinyl formal monomers of the following structure,

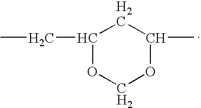

Other mechanisms of hydrogel preparation involve physical crosslinking due to crystallite formation (e.g., due to freeze-thaw processing) and chemical crosslinking using ionizing radiation such as electron-beam and gamma-ray irradiation. These methods may in some instances be advantageous over techniques that employ chemical cross-linking agents, because they do not leave behind non-reacted chemical species.

In a specific example, porous polyol spheres may be formed as described in Pub. No. US 2003/0185895 to Lanphere et al. Briefly, a solution containing a polyol such as PVA and a gelling precursor such as sodium alginate may be delivered to a viscosity controller, which heats the solution to reduce its viscosity prior to delivery to a droplet generator. The droplet generator forms and directs drops into a gelling solution containing a gelling agent which interacts with the gelling precursor. For example, in the case where an alginate gelling precursor is employed, an agent containing a divalent metal cation such as calcium chloride may be used as a gelling agent, which stabilizes the drops by gel formation based on ionic crosslinking. The concentration of the gelling agent can control void formation in the particle, thereby controlling the porosity gradient in the particle. Adding non-gelling ions, for example, sodium ions, to the gelling solution can limit the porosity gradient, resulting in a more uniform intermediate porosity throughout the particle. The gel-stabilized drops may then be transferred to a reactor vessel where the polymer in the gel-stabilized drops reacted, thereby forming precursor particles. For example, the reactor vessel may include an agent that chemically reacts with the polyol to cause interchain or intrachain crosslinking. For instance, the vessel may include an aldehyde and an acid, leading to acetalization of the polyol. The precursor particles are then transferred to a gel dissolution chamber, where the gel is dissolved. For example, ionically crosslinked alginate may be removed by ion exchange with a solution of sodium hexa-metaphosphate. Alginate may also be removed by radiation degradation. Porosity is generated due to the presence (and ultimate removal) of the alginate. The particles may then be filtered to remove any residual debris and to sort the particles into desired size ranges.

Using the above and other techniques, porous particles may be formed having a variety of diameters, pore sizes and porosities. Moreover, porous acetalized PVA particles are commercially available (e.g., as Contour SE™ embolic agent, Boston Scientific, Natick, Mass., USA).

Once suitable polymeric particles are obtained, in accordance with an aspect of the invention, the particles may be loaded with one or more active agents. In other aspects, one or more active agents are incorporated into the polymers that are used to form polymeric particles in accordance with the invention (e.g., via covalent attachment along the backbones or the ends of the polymers). In other aspects, active agents incorporated into the particles of the invention at the time of particle formation (e.g., by blending the active agents with the polymers prior to particle formation, etc.)

In one method, polymeric particles are exposed to a solution containing one or more active agents. To increase solution uptake, the polymeric particles may be dried by any suitable method, including lyophilization (freeze drying). Using dry particles, solution uptake is enhanced, much like a dry sponge is able to absorb more liquid than a wet sponge. Depending on the nature of the polymeric particles and the therapeutic agents, the solvent systems used to create the solution may be based on (a) water, (b) one or more organic solvents, or (c) water and one or more organic solvents. Typically, the one or more active agents should be soluble in the selected solvent system. Furthermore, the selected solvent system should not destroy the integrity of the polymeric particles. In some embodiments, a solvent system is selected that swells the particles to some degree. In those specific embodiments where the polymeric particles are hydrogels, the solvent system may be, for example, based upon water, upon one or more polar organic solvents (e.g., ethanol), or upon water plus one or more polar organic solvents. Polar organic solvents may be used, for example, in conjunction with the loading of more hydrophobic active agents.

The particles of the invention may be stored and transported in dry form. The dry composition may also optionally contain additional agents, for example, one or more of the following among others: (a) tonicity adjusting agents including sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), (b) suspension agents including various surfactants, wetting agents, and polymers (e.g., albumen, PEO, polyvinyl alcohol, block copolymers, etc.), (c) imaging contrast agents (e.g., Omnipaque™, Visipaque™, etc.), and (d) pH adjusting agents including various buffer solutes. The dry composition may shipped, for example, in a syringe, catheter, vial, ampoule, or other container, and it may be mixed with an appropriate liquid carrier (e.g. sterile water for injection, physiological saline, phosphate buffer, a solution containing an imaging contrast agent, etc.) prior to administration. In this way the concentration of the composition to be injected may be varied at will, depending on the specific application at hand, as desired by the health care practitioner in charge of the procedure. One or more containers of liquid carrier may also be supplied and shipped, along with the dry particles, in the form of a kit.

The embolic particles may also be stored in a suspension that contains water in addition to the particles themselves, as well as other optional agents such as one or more of the tonicity adjusting agents, suspension agents, contrast media, and pH adjusting agents listed above, among others. The suspension may be stored, for example, in a syringe, catheter, vial, ampoule, or other container. The suspension may also be mixed with a suitable liquid carrier (e.g. sterile water for injection, physiological saline, phosphate buffer, a solution containing contrast agent, etc.) prior to administration, allowing the concentration of administered particles (as well as other optional agents) in the suspension to be reduced prior to injection, if so desired by the health care practitioner in charge of the procedure. One or more containers of liquid carrier may also be supplied to form a kit.

The amount of embolic particles within a suspension to be injected may be determined by those of ordinary skill in the art. The amount of particles may be limited by the fact that when the amount of particles in the composition is too low, too much liquid may be injected, possibly allowing particles to stray far from the site of injection, which may result in undesired embolization or bulking of vital organs and tissues. When the amount of particles is too great, the delivery device (e.g., catheter, syringe, etc.) may become clogged.

As noted above, permanent or temporary occlusion of blood vessels is useful for managing various diseases, disorders and conditions, including tumors.

For example, fibroids, also known as leiomyoma, leiomyomata or fibromyoma, are the most common benign tumors of the uterus. These non-cancerous growths are present in significant fraction of women over the age of 35. In most cases, multiple fibroids are present, often up to 50 or more. Fibroids can grow, for example, within the uterine wall (“intramural” type), on the outside of he uterus (“subserosal” type), inside the uterine cavity (“submucosal” type), between the layers of broad ligament supporting the uterus (“interligamentous” type), attached to another organ (“parasitic” type), or on a mushroom-like stalk (“pedunculated” type). Fibroids may range widely in size, for example, from a few millimeters to 40 centimeters. In some women, fibroids can become enlarged and cause excessive bleeding and pain. While fibroids have been treated in the past by surgical removal of the fibroids (myomectomy) or by removal of the uterus (hysterectomy), recent advances in uterine embolization now offer a nonsurgical treatment. Thus, embolic particles in accordance with the present invention can be used to treat uterine fibroids.

Methods for treatment of fibroids by embolization are well known to those skilled in the art (see, e.g., Pub. No. US 2003/0206864 to Mangin and the references cited therein). Uterine embolization is aimed at starving fibroids of nutrients. Numerous branches of the uterine artery may supply uterine fibroids. In the treatment of fibroids, embolization of the entire uterine arterial distribution network is often preferred. This is because it is difficult to selectively catheterize individual vessels supplying only fibroids, the major reason being that there are too many branches for catheterization and embolization to be performed in an efficient and timely manner. Also, it is difficult to tell whether any one vessel supplies fibroids rather than normal myometrium. In many women, the fibroids of the uterus are diffuse, and embolization of the entire uterine arterial distribution affords a global treatment for every fibroid in the uterus.

In a typical procedure, a catheter is inserted near the uterine artery by the physician (e.g., with the assistance of a guide wire). Once the catheter is in place, the guide wire is removed and contrast agent is injected into the uterine artery. The patient is then subjected to fluoroscopy or X-rays. In order to create an occlusion, embolic particles are introduced into the uterine artery via catheter. The embolic particles are carried by the blood flow in the uterine artery to the vessels that supply the fibroid. The particles flow into these vessels and clog them, thus disrupting the blood supply to the fibroid. In order for the physician to view and follow the occlusion process, contrast agent may be injected subsequent to infusion of the embolic particles. Treatment is enhanced in the present invention by the one or more active agents that are present in the particles.

Controlled, selective obliteration of the blood supply to tumors is also used in treating solid tumors such as renal carcinoma, bone tumor and liver cancer, among various others. The idea behind this treatment is that preferential blood flow toward a tumor will carry the embolic particles to the tumor thereby blocking the flow of blood which supplies nutrients to the tumor, thus, causing it to shrink. Embolization may be conducted as an enhancement to chemotherapy or radiation therapy. Treatment is enhanced in the present invention by the one or more active agents that are present in the particles.

The present invention encompasses various ways of administering the particulate compositions of the invention to effect embolization. One skilled in the art can determine the most desirable way of administering the particles depending on the type of treatment and the condition of the patient, among other factors. Methods of administration include, for example, percutaneous techniques as well as other effective routes of administration. For example, the embolic particles of the invention may be delivered through a syringe or through a catheter, for instance, a FasTracker® microcatheter (Boston Scientific, Natick, Mass., USA), which can be advanced over a guidewire, a steerable microcatheter, or a flow-directed microcatheter (MAGIC, Balt, Montomorency, France).

Various aspects of the invention of the invention relating to the above are enumerated in the following paragraphs:

Aspect 1. A medical article comprising an active agent, wherein upon implantation or insertion into the vasculature of a subject, said active agent (a) decreases the level of nutrients in the local environment, (b) inhibits the utilization of nutrients by the tumor cells, (c) increases the level of molecular oxygen in the local environment, (d) decreases the level of molecular oxygen in the local environment, (e) increases the level of reactive oxygen species in the local environment, (f) decreases the level of reactive oxygen species in the local environment, or a combination of two or more of the foregoing effects.

Aspect 2. The medical article of aspect 1, wherein the medical article is selected from a stent, a catheter, a vascular graft, an occlusion balloon, and an embolic implant.

Aspect 3. The medical article of aspect 1, wherein the medical article is an embolic implant selected from an embolic particle and an embolic coil.

Aspect 4. The medical article of aspect 1, wherein the active agent is releasably or non-releasably disposed on a surface of the medical article.

Aspect 5. The medical article of aspect 1, wherein the active agent is releasably or non-releasably disposed within a material of the medical article.

Aspect 6. The medical article of aspect 1, wherein the medical article comprises a polymeric, metallic or ceramic material.

Aspect 7. The medical article of aspect 1, wherein the medical article comprises a polymeric material that comprises one or more of the following monomers: vinyl alcohol, vinyl formal.

Aspect 8. The medical article of aspect 1, wherein the active agent is selected from active agents that bind amino acid containing nutrients, active agents that bind carbohydrate containing nutrients, active agents that bind lipid containing nutrients and active agents that bind metallic nutrients.

Aspect 9. The medical article of aspect 8, wherein the active agent is selected from protein binding ligands, protein coagulants, carbohydrate binding ligands, positively charged ion exchange groups, and chelating groups.

Aspect 10. The medical article of aspect 8, wherein the active agent acts to bind the nutrients to the medical article or the active agent is released from the medical article to bind the nutrients outside the medical article.

Aspect 11. The medical article of aspect 1, further comprising an additional agent selected from a chemotherapy agent and a vasoactive agent.

Aspect 12. The medical article of aspect 1, wherein the active agent is an oxygen scavenging agent that decreases the level of molecular oxygen in the local environment.

Aspect 13. The medical article of aspect 12, wherein the active agent is a photosensitizing agent that scavenges oxygen upon exposure to photons of a suitable wavelength and produces a reactive oxygen species that is toxic to cells.

Aspect 14. The medical article of aspect 12, further comprising an additional active agent that undergoes bioreductive activation under hypoxic conditions to produce a species that is toxic to cells.

Aspect 15. The medical article of aspect 14, wherein the additional active agent is selected from nitroimidazoles, quinones, and N-oxides.

Aspect 16. The medical article of aspect 1, wherein the active agent is an agent that increases the level of molecular oxygen in the local environment.

Aspect 17. The medical article of aspect 1, wherein the active agent is selected from perfluorocarbon and hemoglobin species.

Aspect 18. The medical article of aspect 1, wherein the active agent decreases the level of reactive oxygen species in the local environment.

Aspect 19. The medical article of aspect 1, wherein the active agent increases the level of reactive oxygen species in the local environment.

Aspect 20. The medical article of aspect 19, wherein the active agent is selected from agents that release reactive oxygen species in vivo and agents that interfere with the subject's ability to scavenge reactive oxygen species.

Aspect 21. The medical article of aspect 1, wherein the active agent inhibits the utilization of nutrients by the tumor cells.

Aspect 22. The medical article of aspect 21, wherein the active agent is an antimetabolite.

Aspect 23. A method of treatment comprising inserting or implanting the medical article of aspect 1 into the vasculature of a subject.

Aspect 24. The method of aspect 23, further comprising administering total parenteral nutrition to the subject.

Aspect 25. A method of treatment comprising (a) determining the molecular oxygen level within the vasculature of a tumor of a subject and (b) inserting or implanting a medical article into the tumor vasculature, wherein the medical article comprises an active agent that causes an increase or a decrease the level of molecular oxygen in the local environment upon implantation or insertion.

Aspect 26. A method of treatment comprising (a) inserting or implanting a first medical article into the vasculature of a tumor of a subject, wherein the first medical article comprises an active agent that causes an increase in the level of molecular oxygen in the local environment upon implantation or insertion, and (b) inserting or implanting a second medical article into said vasculature, wherein the second medical article comprises an active agent that causes a decrease in the level of molecular oxygen in the local environment upon implantation or insertion, wherein step (a) may be a predecessor or successor of step (b).

Aspect 27. The method of aspect 26, further comprising subjecting the subject to chemotherapy or radiation therapy concurrent with or subsequent to step (a).

Aspect 28. A method of treatment comprising (a) inserting or implanting one or more medical articles into the vasculature of a tumor of a subject, wherein the one or more medical articles comprise (i) a photosensitizing agent that scavenges oxygen upon exposure to photons of a particular wavelength and produces a reactive oxygen species that is toxic to cells and (ii) an agent that increases the level of molecular oxygen in the local environment, and (b) exposing the one or more medical articles to said photons.

Although various embodiments are specifically illustrated and described herein, it will be appreciated that modifications and variations of the present invention are covered by the above teachings and are within the purview of any appended claims without departing from the spirit and intended scope of the invention.

1. A medical article comprising an active agent, wherein upon implantation or insertion into the vasculature of a subject, said active agent (a) decreases the level of nutrients in the local environment, (b) inhibits the utilization of nutrients by the tumor cells, (c) increases the level of molecular oxygen in the local environment, (d) decreases the level of molecular oxygen in the local environment, (e) increases the level of reactive oxygen species in the local environment, (f) decreases the level of reactive oxygen species in the local environment, or a combination of two or more of the foregoing effects.

2. The medical article of claim 1, wherein said medical article is selected from a stent, a catheter, a vascular graft, an occlusion balloon, and an embolic implant.

3. The medical article of claim 1, wherein said medical article is an embolic implant selected from an embolic particle and an embolic coil.

4. The medical article of claim 1, wherein said active agent is releasably or non-releasably disposed on a surface of the medical article.

5. The medical article of claim 1, wherein said active agent is releasably or non-releasably disposed within a material of the medical article.

6. The medical article of claim 1, wherein said medical article comprises a polymeric, metallic or ceramic material.

7. The medical article of claim 1, wherein said medical article comprises a polymeric material that comprises one or more of the following monomers: vinyl alcohol, vinyl formal.

8. The medical article of claim 1, wherein said active agent is selected from active agents that bind amino acid containing nutrients, active agents that bind carbohydrate containing nutrients, active agents that bind lipid containing nutrients and active agents that bind metallic nutrients.

9. The medical article of claim 8, wherein said active agent is selected from protein binding ligands, protein coagulants, carbohydrate binding ligands, positively charged ion exchange groups, and chelating groups.

10. The medical article of claim 8, wherein said active agent acts to bind the nutrients to the medical article or the active agent is released from the medical article to bind the nutrients outside the medical article.

11. The medical article of claim 1, further comprising an additional agent selected from a chemotherapy agent and a vasoactive agent.

12. The medical article of claim 1, wherein said active agent is an oxygen scavenging agent that decreases the level of molecular oxygen in the local environment.

13. The medical article of claim 12, wherein said active agent is a photosensitizing agent that scavenges oxygen upon exposure to photons of a suitable wavelength and produces a reactive oxygen species that is toxic to cells.

14. The medical article of claim 12, further comprising an additional active agent that undergoes bioreductive activation under hypoxic conditions to produce a species that is toxic to cells.

15. The medical article of claim 14, wherein said additional active agent is selected from nitroimidazoles, quinones, and N-oxides.

16. The medical article of claim 1, wherein said active agent is an agent that increases the level of molecular oxygen in the local environment.

17. The medical article of claim 1, wherein said active agent is selected from perfluorocarbon and hemoglobin species.

18. The medical article of claim 1, wherein said active agent decreases the level of reactive oxygen species in the local environment.

19. The medical article of claim 1, wherein said active agent increases the level of reactive oxygen species in the local environment.

20. The medical article of claim 19, wherein said active agent is selected from agents that release reactive oxygen species in vivo and agents that interfere with the subject's ability to scavenge reactive oxygen species.

21. The medical article of claim 1, wherein said active agent inhibits the utilization of nutrients by the tumor cells.

22. The medical article of claim 21, wherein said active agent is an antimetabolite.

23. A method of treatment comprising inserting or implanting the medical article of claim 1 into the vasculature of a subject.

24. The method of claim 23, further comprising administering total parenteral nutrition to the subject.

25. A method of treatment comprising (a) determining the molecular oxygen level within the vasculature of a tumor of a subject and (b) inserting or implanting a medical article into the tumor vasculature, wherein the medical article comprises an active agent that causes an increase or a decrease the level of molecular oxygen in the local environment upon implantation or insertion.

26. A method of treatment comprising (a) inserting or implanting a first medical article into the vasculature of a tumor of a subject, wherein the first medical article comprises an active agent that causes an increase in the level of molecular oxygen in the local environment upon implantation or insertion, and (b) inserting or implanting a second medical article into said vasculature, wherein the second medical article comprises an active agent that causes a decrease in the level of molecular oxygen in the local environment upon implantation or insertion, wherein step (a) may be a predecessor or successor of step (b).

27. The method of claim 26, further comprising subjecting said subject to chemotherapy or radiation therapy concurrent with or subsequent to step (a).

28. A method of treatment comprising (a) inserting or implanting one or more medical articles into the vasculature of a tumor of a subject, wherein the one or more medical articles comprise (i) a photosensitizing agent that scavenges oxygen upon exposure to photons of a particular wavelength and produces a reactive oxygen species that is toxic to cells and (ii) an agent that increases the level of molecular oxygen in the local environment, and (b) exposing the one or more medical articles to said photons.
